(19) United States
(12) Patent Application Publication
(10) Pub. No.: US 2004/0216845 A1
(43) Pub. Date: Nov. 4, 2004

(54) Non-thermal plasma generator device

(76) Inventor: Czeslaw Golkowski, Ithaca, NY (US)

Correspondence Address: HANCOCK & ESTABROOK, LLP, 1500 MONY Tower I, PO Box 4976, Syracuse, NY, 13221-4976, US

(21) Appl. No.: 10/429,542

(22) Filed: May 2, 2003

(51) Int. Cl. C23F 1/00

(52) U.S. Cl. 156/345.41

(57) ABSTRACT

A non-thermal plasma generation device for non-destructively decontaminating sensitive surfaces, such as living tissue, electronic equipment and other surfaces that cannot tolerate high temperatures or aggressive chemicals, using the free radicals and excited states of gas produced in an atmospheric-pressure air plasma. The plasma is preferably generated by a stable, self-igniting discharge in a resonant waveguide system, driven by a magnetron or other high power microwave source, operating in a pulsed mode.

### BACKGROUND OF THE INVENTION

The present invention relates generally to non-thermal plasma generating devices, and more particularly to such devices used to decontaminate sensitive surfaces, such as living tissue, electronic equipment and other surfaces which cannot tolerate high temperature or aggressive chemicals.

Bacteria, chemicals, and other harmful organisms contaminate sensitive surfaces, such as living tissue, food products, and other surfaces that humans come into contact on a daily basis. It is necessary to decontaminate these surfaces to prevent illness from affecting the humans who may come into contact with the contaminated surface. A common manner of performing the decontamination is to expose the surface to a plasma generated from an inert gas, such as argon, or expose the surface to radiation. While such systems are effective, they are also costly to operate and could adversely affect the surface being treated.

Plasmas have been used in a wide variety of industrial application, such as semiconductor fabrication and coatings of reflective films for window panels and compact discs. The principal advantage of plasma cleaning is that it is an “all dry” process, generates minimal effluent, does not require hazardous pressures, and is applicable to a wide variety of vacuum compatible materials, including silicon, metals, glass, and ceramics.

U.S. Pat. No. 5,961,772 to Selwyn describes an atmospheric, non-thermal plasma jet which produces metastable and reactive species that are useful for etching and cleaning surfaces. The Background section of the '772 patent describes the benefits of and the state of the art in non-thermal, atmospheric plasmas in great detail, and with the exception of that invention's objects and advantages, its background section is hereby incorporated by reference.

It is a principal object and advantage of the present invention to provide a non-thermal, atmospheric plasma generating device that may be used to decontaminate sensitive surfaces and that is inexpensive to operate.

It is a further object and advantage of the present invention to provide a plasma generating device that is non-harmful to sensitive surfaces.

It is an additional object and advantage of the present invention to provide a plasma generating device that produces plasma at temperatures conducive to contact human skin.

Other objects and advantages of the present invention will in part be obvious, and in part appear hereinafter.

### SUMMARY OF THE INVENTION

In accordance with the foregoing objects and advantages the present invention provides a plasma generation device for non-destructive decontamination of sensitive surfaces, comprising a waveguide of predetermined length extending between first and second opposite ends and including a cavity in which waves may propagate; an electromagnetic wave generator connected to the waveguide, adjacent its first end, for generating waves of electromagnetic energy having predetermined wavelengths in the waveguide; a gas conduit extending along a longitudinal axis and positioned in fluid communication with the cavity and at a predetermined distance from the second end where the electric field is particularly strong; a plasma initiating device in fluid communication with the gas conduit; and an exit port formed through the waveguide in fluid communication with said gas conduit through which the plasma of metastable and excited state of gas may flow. A sensitive surface a predetermined distance beneath the exit port will be impacted by the plasma which, in turn, will kill certain types of bacteria present thereon.

### BRIEF DESCRIPTION OF THE DRAWINGS

The present invention will be more fully understood and appreciated by reading the following Detailed Description in conjunction with the accompanying drawings, in which:

FIG. 1 is a perspective view of the present invention with a portion broken away;

FIG. 2 is an exploded perspective view of the gas introduction and plasma generation system of the present invention;

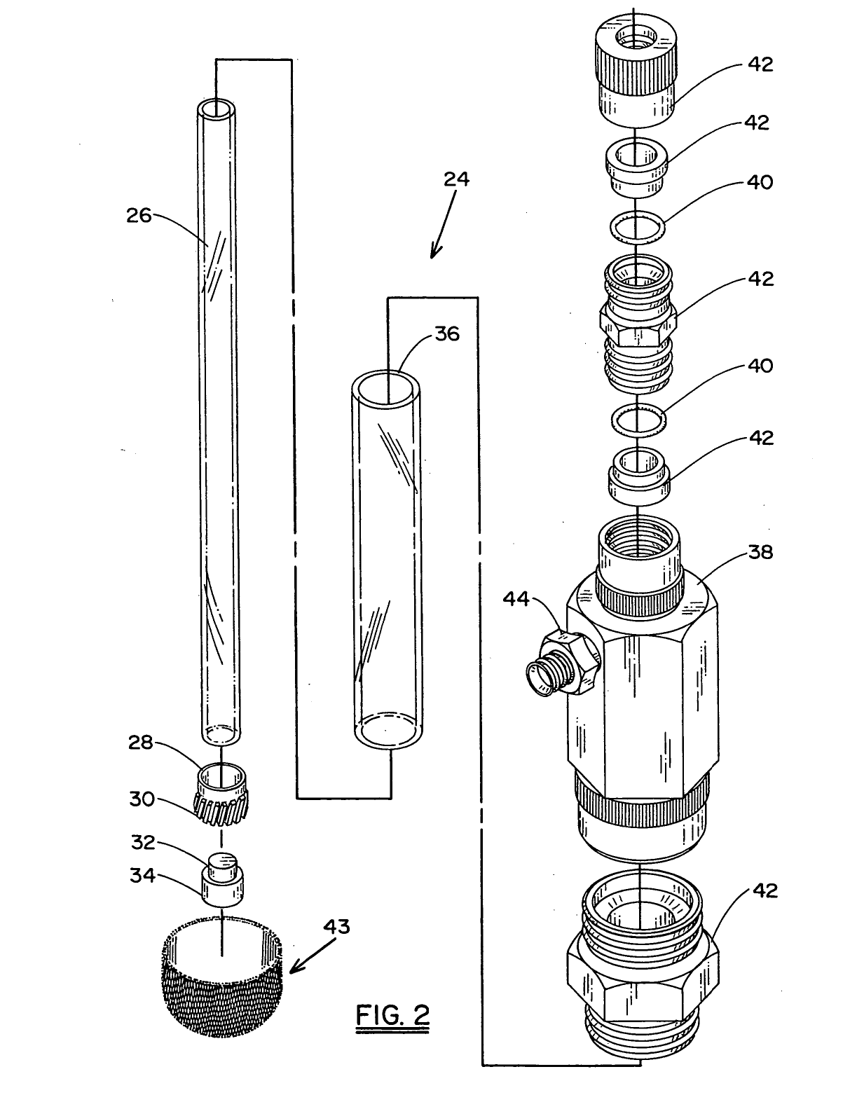

FIG. 3 is a cross-sectional view taken along line 3-3 of FIG. 1; and

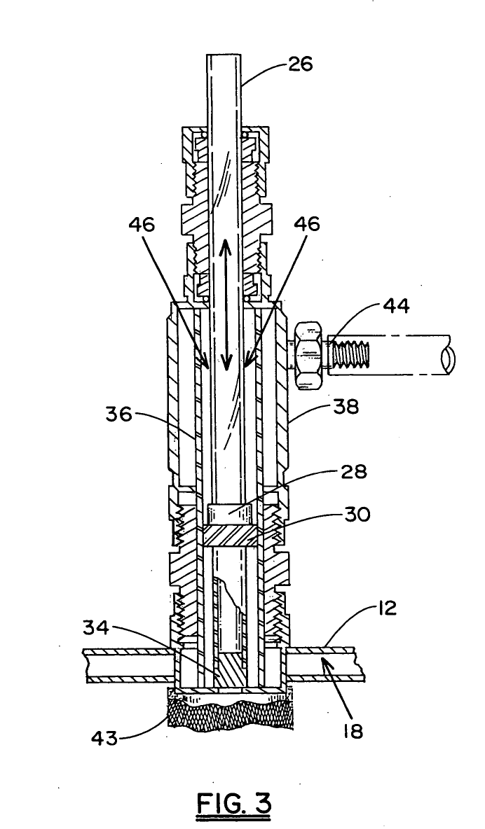

FIG. 4 is a front elevation view of the present invention with a field strength plot.

### DETAILED DESCRIPTION OF A PREFERRED EMBODIMENT

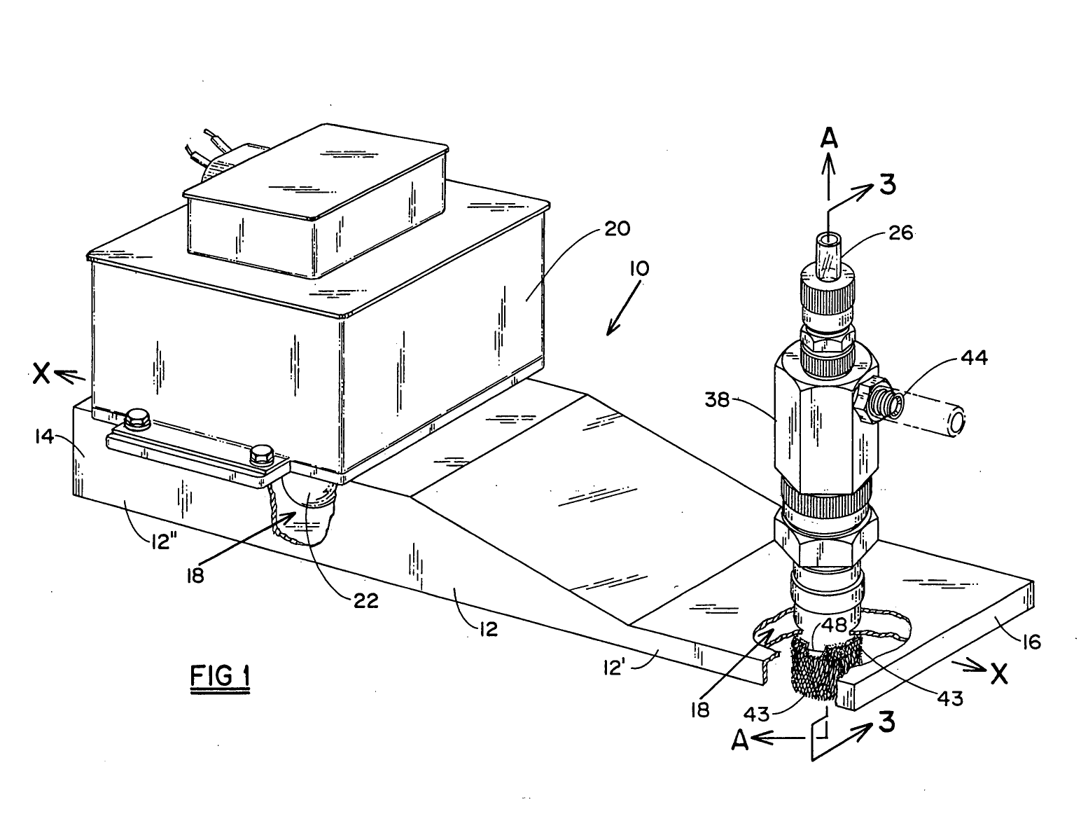

Referring now to the drawings, in which like reference numerals refer to like parts throughout, there is seen in FIG. 1 a plasma generation device, designated generally by reference numeral 10, for non-destructively decontaminating sensitive surfaces, such as living tissue, electronic equipment and other surfaces that cannot tolerate high temperatures or aggressive chemicals, using the free radicals and excited states of gas produced in an atmospheric-pressure air plasma. As will be described in greater detail hereinafter, the plasma is preferably generated by a stable, self-igniting discharge in a resonant waveguide system, driven by a magnetron or other high power microwave source, operating in a pulsed mode.

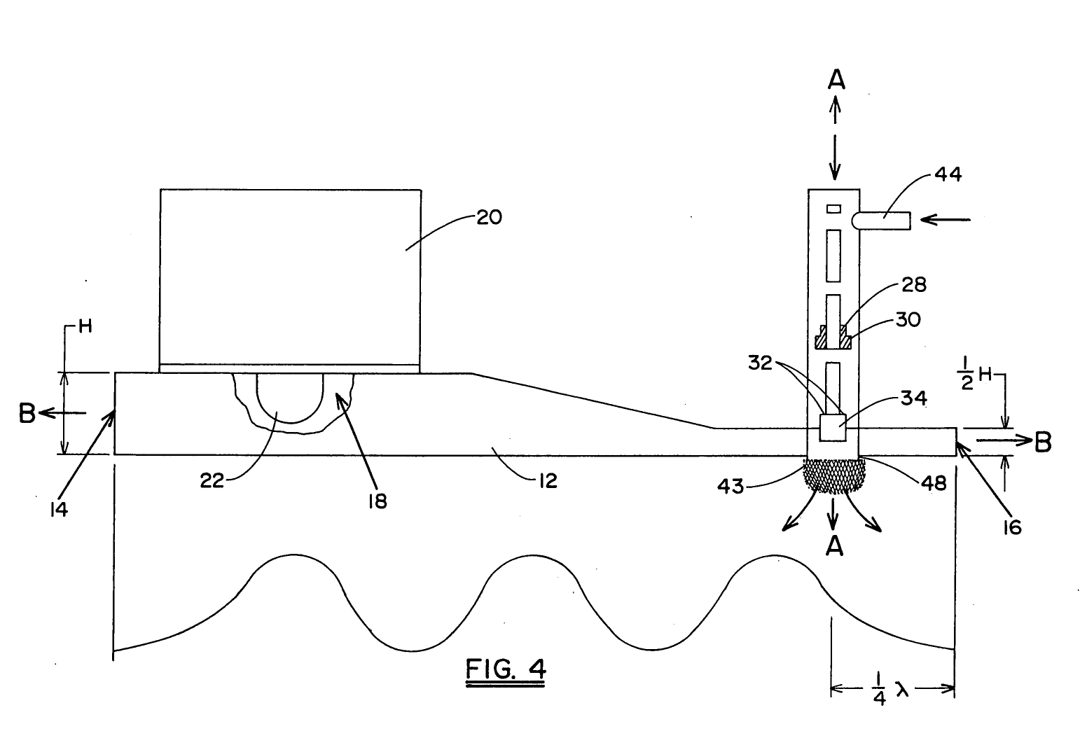

Plasma generation device 10 generally comprises a waveguide 12 extending a predetermined length L between closed, opposing ends 14, 16 along a longitudinal axis X-X, and defining a resonant cavity 18 in which waves may propagate, a magnetron (or equivalent high energy power microwave source capable of producing electromagnetic waves) 20 positioned adjacent end 14 and having its electromagnetic wave generating portion 22 positioned within cavity 18, and a gas introduction and plasma discharge system, designated generally by reference numeral 24, positioned in fluid communication with cavity 18 and a predetermined distance from end 16, preferably ¼ wavelength (see FIG. 4) for purposes that will be explained hereinafter.

Gas introduction and plasma discharge system 24 essentially comprises an inner tube 26 that is used to support a ring 28 that includes a plurality of vanes 30 formed on its outer surface and a conductive element 32 having a lower portion 34 that is preferably terminates in a sharp edge, and an outer tube 36 concentrically positioned in spaced relation around inner tube 26. Ring 28 and vanes 30 are positioned between inner tube 26 and outer tube 36. Gas introduction and plasma discharge system 24 further comprises a housing 38 that extends through waveguide 12 and into cavity 18, and a series of sealing rings 40 and tube supports 42 that maintain outer tube 36 and inner tube 26 in sealed, concentric position within housing 38. Tubes 26, 36 co-axially extend along a longitudinal axis A-A that is essentially transverse to the longitudinal axis X-X along which waveguide 12 extends, and is positioned in an area of high electric field sufficient to establish an electric breakdown of air or other gas at atmospheric pressure. For the preferred embodiment of the invention, waveguide 12 is about 2 wavelengths long and includes a working end 12′ that is about, but no more than ½ the height H of the wave generating end 12″, as illustrated in FIG. 4, and the electric field generated by magnetron 12 is about 30 kV/cm and the position where tubes 26, 36 should be positioned is about a ¼ wavelength from end 16. Also, discharge system may further include a conductive mesh 43 at its outlet. Mesh 43 prevents any dissipation of microwaves.

Housing 38 further includes an auxiliary port 44 extending through a sidewall thereof and through which an auxiliary gas or atomized liquid additives may be introduced if desired. For instance, atomized liquid bleach may be introduced into the air stream where it is mixed via vanes 30 to act as an additional agent to kill off any bacteria.

In operation, compressed air or other gas mixture is introduced into gas introduction and plasma discharge system 24, and specifically through the top of the area separating inner tube 26 and outer tube 36, as indicated by arrow 46. The air flows downwardly through gas introduction and plasma discharge system 24 and is mixed with any additives injected through auxiliary port 44 and forces the mixture downwardly. The air and/or mixture of air and additives are then mixed into a turbulent state by passing through vanes 30 which produces a uniform cross-section, stable plasma. The air and/or mixture pass over conductive element 32 that provides free electrons which, coupled with the electric field, produces an effluent that exits through a port 48 formed through the bottom wall of waveguide 12. The effluent of metastable and excited states of air/gas molecules and whatever additives may have been introduced extends a few centimeters away from port 48, although its maximum effects are present in about the first centimeter of discharge.

Recent tests utilizing the above described device have shown the following results:

Destruction of Volatile Organic Compounds (VOC) in air with varying pulse width within a range 1-2.5 microsecond and repetition rate 20,000 pps. The removal of 100% of propane in air was achieved for the flow rate of 60 slm (standard liters per minute) with the following output gas temperature:

temp. ˜80° C., 100% removal up to 1,000 ppm (parts per million)

temp. ˜70° C., 100% removal up to 500 ppm

temp. ˜50° C., 100% removal up to ˜10 ppm

The present invention has been described with particular reference to a preferred embodiment and a best mode for practicing the invention. It is contemplated, however, that changes may be made to this preferred embodiment without departing from the true spirit and scope of the present invention that is defined by the appended claims.

What is claimed is:

1. A plasma generation device for non-destructive decontamination of sensitive surfaces, comprising:
a. a waveguide of predetermined length extending between first and second opposite ends and including a cavity in which waves may propogatepropagate;
b. an electromagnetic wave generator connected to said waveguide, adjacent its said first end, for generating waves of electromagnetic energy having predetermined wavelengths in said waveguide;
c. a gas conduit extending along a longitudinal axis and positioned in fluid communication with said cavity and at a predetermined distance from said second end;
d. a plasma initiating device in fluid communication with said gas conduit; and
e. an exit port formed through said waveguide in fluid communication with said gas conduit.

2. The plasma generation device according to claim 1, wherein said waveguide is rectangular in cross-section.

3. The plasma generation device according to claim 2, wherein said waveguide is resonant.

4. The plasma generation device according to claim 1, wherein said electromagnetic wave generator is a pulsed magnetron.

5. The plasma generation device according to claim 4, wherein said pulsed magnetron operates a microwave frequency.

6. The plasma generation device according to claim 1, wherein said gas conduit is a quartz tube.

7. The plasma generation device according to claim 1, wherein said plasma initiating device comprises a conductive ring positioned within said gas conduit.

8. The plasma generation device according to claim 1, wherein said predetermined distance said gas conduit is positioned from said second end is about ¼ of a said wavelength.

9. The plasma generation device according to claim 1, wherein said gas conduit includes an auxiliary port formed therethrough.

10. The plasma generation device according to claim 1, further comprising a conductive mesh engaged with said exit port.
